(19) United States
(12) Patent Application Publication
(10) Pub. No.: US 2018/0036160 A1
(43) Pub. Date: Feb. 8, 2018

(54) 4heelpain

(71) Applicant: Russell Ellicott, Augusta, GA (US)

(72) Inventor: Russell Ellicott, Augusta, GA (US)

(21) Appl. No.: 15/229,298

(22) Filed: Aug. 5, 2016

(51) Int. Cl. A61F 5/01; A61F 5/14; A41B 11/02

(52) U.S. Cl. CPC A61F 5/0111; A41B 11/02; A61F 5/14

(57) ABSTRACT

4heelpain includes a T-shaped device for treatment of heel pain. The upper portion of the “T” consists of one or two layers of elastic material and foam padding. The upper portion of the “T” is secured around the circumference of the superior ankle with Velcro. The lower portion of the “T” consists of one or two layers of elastic material sewn to the upper portion of the “T”. The lower portion of the “T” cradles the heel, is stretched across the heel, and is fastened to the medial side of the upper portion of the “T” with Velcro. A heel wedge made of foam material is placed beneath the heel.

### BACKGROUND OF THE INVENTION

The present invention relates to a device that is stabilized by a superior ankle enclosure that is connected to an adjustable elastic plantar heel area elastic cradle which is accompanied by a heel wedge. The device decreases heel pain by providing continual adjustable compression to the plantar fascia area of the heel and resistance to heel eversion. A heel wedge further resists heel eversion. Compression of the heel along with resistance to heel eversion decreases pain and stress at the plantar fascia of the heel.

### DESCRIPTION OF THE PRIOR ART

Human heel pain results, in the great majority of the time, from injury to the plantar fascia tissue at the bottom of the heel (specifically the area of the calcaneus where the plantar fascia attaches).

The 4heelpain device is unique that it applies adjustable compression across the plantar heel area of the foot to relieve heel pain and resist pronation.

U.S. Pat. No. 9,463,135 by Tabron et al discloses a wrap that surrounds the foot yielding compression to heel and arch with a bladder that contains fluid.

U.S. Pat. No. 9,452,074 by Wynne et al discloses an orthotic device designed to provide force to resist motion of a body part.

U.S. Pat. No. 9,439,457 by Baravarian discloses an arch support sock with varying compressions applied to different areas of the foot and arch.

U.S. Pat. No. 5,067,486 by Hely discloses an ankle stabilizing brace composed of straps that encompass the foot.

As noted by the above patents, several devices have been utilized to stabilize the ankle, compress the heel, or resist motion of body parts. No device has been found to exist the provides direct compression of the heel while resisting the forces of heel eversion in the manner of direct elastic compression of the heel.

### SUMMARY OF THE INVENTION

The present invention relates to a device called 4heelpain which consists of an elastic ankle collar surrounding foam, an elastic strap that is attached to the ankle collar and cradles the heel, and a foam wedge that is placed beneath the heel. The device is adjusted by stretching the elastic around the ankle and heel and attaching it with Velcro that is sewn to the ends of the elastic. The 4heelpain device when installed properly applies pressure to the heel and opposes eversion of the heel.

It is therefore the object of the 4heelpain device to provide compression to the injured, painful heel and resists heel eversion.

The advantages and features of the 4heelpain device should become more apparent upon review of the detailed description and drawings of the 4heelpain device.

### BRIEF DESCRIPTION OF THE 4HEELPAIN DEVICE

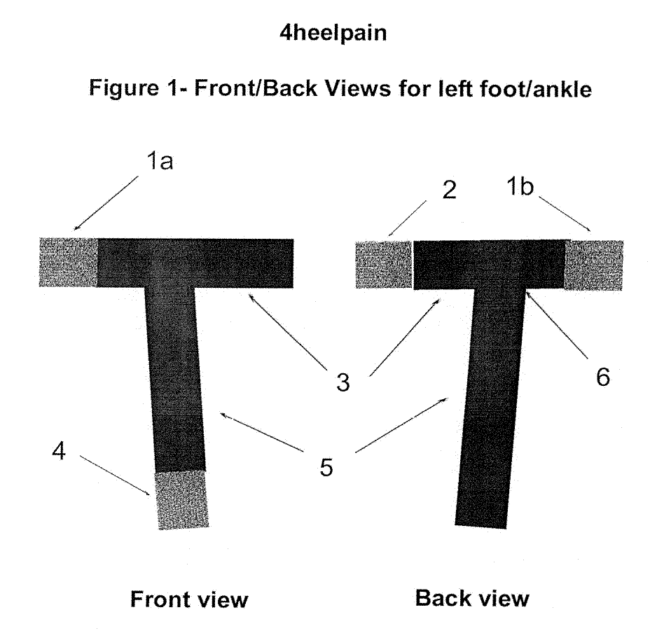

FIG. 1 is a front and rear view of the elastic ankle collar sewn to the elastic that cradles the heel which is composed of elastic and Velcro

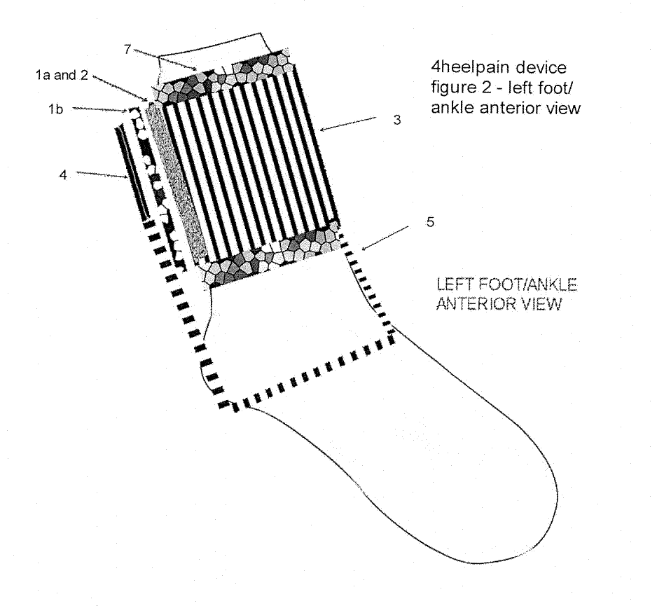

FIG. 2 is a left anterior view of the 4heelpain elastic ankle collar surrounding the superior area of a left ankle that is surrounded by foam that surrounds a sock over the foot and ankle. An elastic strap is stretched laterally and cradles the heel to be attached medially.

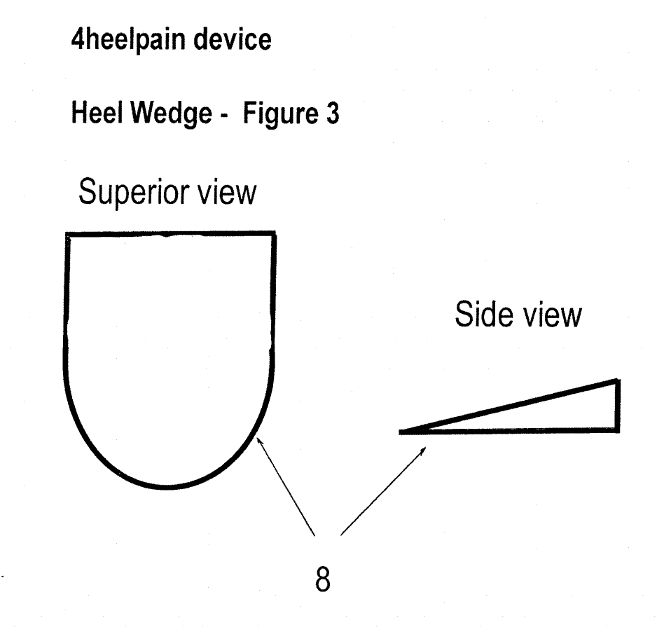

FIG. 3 is superior and lateral views of the dense foam heel wedge

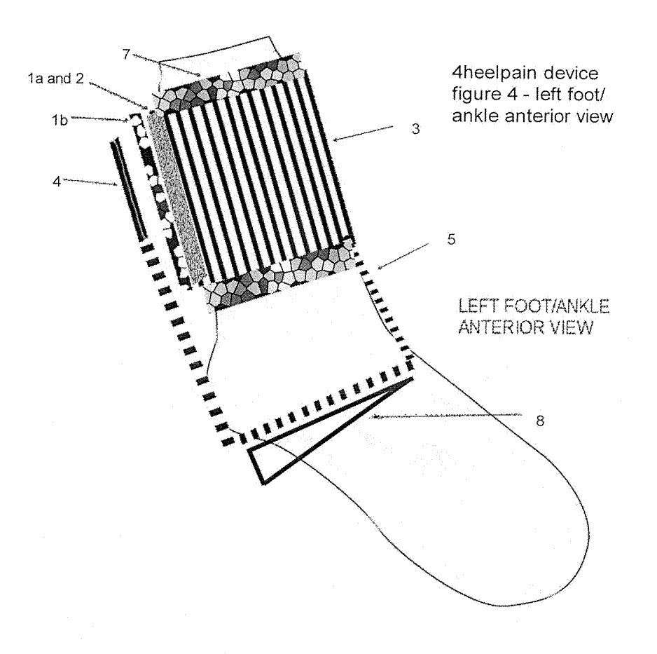

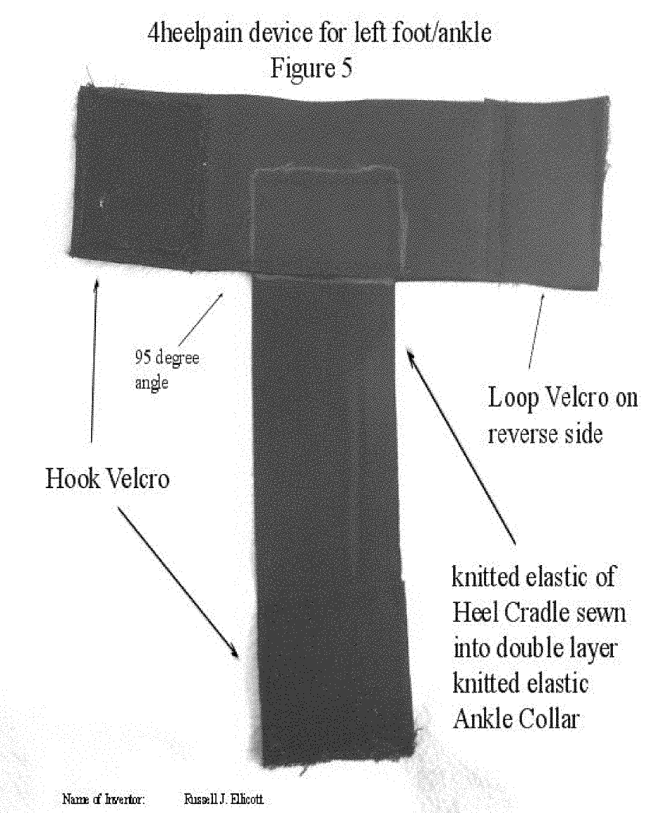

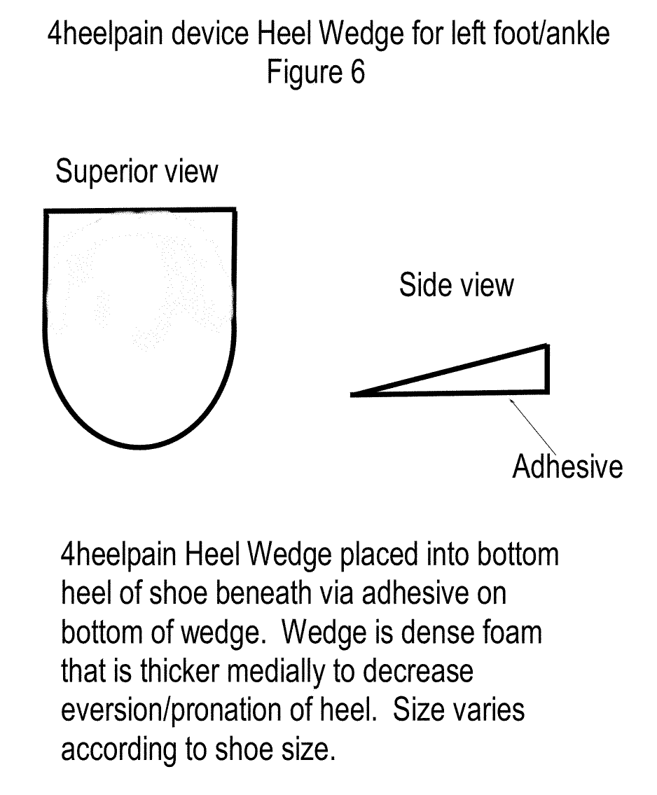

FIG. 4 is FIG. 2 with the addition of the heel wedge seen in FIG. 3.

### DESCRIPTION OF THE PREFERRED EMBODIMENT

Please refer to FIGS. 1, 2, 3, and 4 of 4heelpain device. The sizes of the components described below are for an adult male with a US shoe size of 10-12. Sizes of material involved vary to reflect the individual person the 4heelpain device would be applied to.

Item labeled 1a consists of Hook Velcro, 3″ wide, 3″ long, sewn to Item labeled 3

Item labeled 1b consists of Loop Velcro, 3″ wide, 3″ long, sewn to Item labeled 3

Item labeled 2 consists of Loop Velcro, 3″ wide, 3″ long, sewn to Item labeled 3

Item labeled 3 consists of single or double layered knitted elastic, 3″ wide, 11″ non-stretched length, sewn to Item labeled 5

Item labeled 4 consists of Hook Velcro, 3″ wide, 3″ long, sewn to Item labeled 3

Item labeled 5 consists of single or double layered knitted elastic, 3″ wide, 9″ non-stretched length, sewn to Item labeled 3

Item labeled 6 represents a 95 degree angle between Item labeled 3 and Item labeled 5

Item labeled 7 consists of dense foam, 5″ wide, 12″ long, placed beneath Items labeled 1a, 1b, 2, and 3

Item labeled 8 consists of dense foam shaped as the heel of a shoe, 3″ wide, 3″ long, ⅜″ medial side depth that tapers to less than 1/16″ depth laterally

Item labeled 3 is stretched over Item labeled 7 with inferior border of Item labeled 3 placed superior to the ankle bone prominences (medial and lateral malleolus). Item labeled 3 is fastened medially by Item labeled 1a that attaches to Item labeled 2, above the medial malleolus. Item labeled 1b is superficial to Item labeled 1a and awaits attachment to Item labeled 4.

Item labeled 5 is centered at the lateral malleolus and is stretched around the heel from lateral to medial, cradling the heel at the plantar fascial attachment to the heel, and is attached to Item labeled 1b by Item labeled 4. Item labeled 5 can be adjusted to increase compression and over the fascial attachment by placing more tension on the stretch of the elastic. Item labeled 5 can vary its angle of stretch as Item labeled 5 passes under the plantar fascial attachment, i.e. Item labeled 4 can vary its attachment position anterior to posterior to Item labeled 1b. Increasing the tension of Item labeled 5 decreases the eversion movement of the heel.

Item labeled 8 is placed plantar to or inferior to Item labeled 5 at the inner bottom heel surface of the shoe with the thicker edge of the wedge placed medially. Item labeled 8 assists the Item labeled 5 to resist eversion of the heel.

All Items can be varied for size, shape, and materials. Tension and attachment of materials can be varied.

What is claimed is:

1. A device to treat heel pain comprising:
An ankle section, composed of elastic and foam material, adjustably stretches around the ankle and fastens superior to the ankle prominences with Hook/Loop Velcro to provide a stable comfortable attachment for the bottom section;

2. a bottom section, composed of elastic, is sewn to the top section and centered laterally, courses from lateral to medial, stretches around the heel and provides continuous adjustable compression to the plantar fascial attachment. The bottom section fastens to the top section on the medial side of the ankle with Velcro. Compression of the heel by the bottom section decreases pain by controlling swelling of the fascia and by supporting the fascia attachment to the heel bone. In addition, tension provided by the bottom section of the device, resists eversion of the heel that allows the foot to naturally decrease the stress on the plantar fascia.

3. The top section has a superficial layer of Loop Velcro sewn to it for attachment to the bottom section Hook Velcro medially;

4. a foam wedge is placed beneath the heel, the wider portion of the wedge placed medially, resists eversion of the heel.
